(19) United States
(12) Patent Application Publication
(10) Pub. No.: US 2005/0216199 A1
(43) Pub. Date: Sep. 29, 2005

(54) Cuffless blood-pressure monitor and accompanying web services interface

(75) Inventor: Matthew J. Banet, Del Mar, CA (US)

Correspondence Address: MATTHEW BANET, 12719 VIA FELINO, DEL MAR, CA, 92014, US

(73) Assignee: TRIAGE DATA NETWORKS

(21) Appl. No.: 10/810,237

(22) Filed: Mar. 26, 2004

(51) Int. Cl. G06F 19/00; G01N 33/48; G01N 33/50; A61B 5/02

(52) U.S. Cl. 702/19; 600/485; 128/903

(57) ABSTRACT

The invention provides a system for monitoring blood pressure that includes: 1) a gateway software system that receives blood pressure information collected with a blood-pressure monitor and transmitted with a wireless interface; 2) a database that receives the blood pressure information from the gateway software system and stores this information or derivatives thereof; and 3) a web services software interface that, in response to a request from a secondary software system, retrieves the blood pressure information or derivative thereof from the database.

### BACKGROUND

1. Field

The present invention relates to a cuffless blood-pressure system and an accompanying web services interface.

2. Description of Related Art

Blood within a patient's body is characterized by a baseline pressure value, called the diastolic pressure. Diastolic pressure indicates a pressure in an artery when the blood it contains is static. A heartbeat forces a time-dependent volume of blood through the artery, causing the baseline pressure to increase in a pulse-like manner to a value called the systolic pressure. The systolic pressure indicates a maximum pressure in a portion of the artery that contains a flowing volume of blood.

Pressure in the artery periodically increases from the diastolic pressure to the systolic pressure in a pulsatile manner, with each pulse corresponding to a single heartbeat. Blood pressure then returns to the diastolic pressure when the flowing pulse of blood passes through the artery.

Both invasive and non-invasive devices can measure a patient's systolic and diastolic blood pressure. A non-invasive medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while the automated sensor monitors blood flow. A microcontroller in the automated device then calculates blood pressure. Cuff-based blood-pressure measurements such as these typically only determine the systolic and diastolic blood pressures; they do not measure dynamic, time-dependent blood pressure.

Time-dependent blood pressure can be measured with an invasive device, called a tonometer. The tonometer is typically inserted into an opening in a patient's skin and features a component that compresses an artery against a portion of bone. A pressure sensor within the device then measures blood pressure in the form of a time-dependent waveform. The waveform features a baseline that indicates the diastolic pressure, and time-dependent pulses, each corresponding to individual heartbeats. The maximum value of each pulse is the systolic pressure. The rising and falling edges of each pulse correspond to pressure values that lie between the systolic and diastolic pressures.

Data indicating blood pressure are most accurately measured during a patient's appointment with a medical professional, such as a doctor or a nurse. Once measured, the medical professional manually records these data in either a written or electronic file. Appointments typically take place a few times each year. Unfortunately, in some cases, patients experience ‘white coat syndrome’ where anxiety during the appointment affects the blood pressure that is measured. For example, white coat syndrome can elevate a patient's heart rate and blood pressure; this, in turn, can lead to an inaccurate diagnoses.

Some medical devices for measuring blood pressure and other vital signs include systems for transmitting data from a remote site, such as the patient's home, to a central database. These systems can include a conventional computer modem that transmits data through a telephone line to the database. Or alternatively they can include a wireless transmitter, such as a cellular telephone, which wirelessly transmits the data through a wireless network.

### BRIEF DESCRIPTION OF THE DRAWINGS

The features and advantages of the present invention can be understood by reference to the following detailed description taken with the drawings, in which:

FIG. 1 is a schematic side view of the cuffless blood-pressure monitor of the invention, featuring a ‘watch’ component and a wireless interface;

FIG. 2 is a schematic view of an Internet-based system including a web services interface, coupled with the blood-pressure monitor of FIG. 1, that transmits blood-pressure data through a wireless network to an Internet-accessible host computer system;

FIG. 3 is a schematic drawing of the web services software interface of FIG. 2, which includes ‘containers’ for both enterprise java beans and web services software;

FIG. 4 is a schematic drawing of the web services software interface of FIG. 3, specifically designed to retrieve blood pressure information for a secondary software system;

FIG. 5 is a screen capture of a web page from the web site of FIG. 1 that plots a patient's time-dependent systolic and diastolic blood pressure; and

FIG. 6 is a screen capture of a web page from the web site of FIG. 1 that plots a patient's time-dependent pulse oximetry information.

### DETAILED DESCRIPTION

The following description refers to the accompanying drawings that illustrate certain embodiments of the present invention. Other embodiments are possible and modifications may be made to the embodiments without departing from the spirit and scope of the invention. Therefore, the following detailed description is not meant to limit the present invention. Rather, the scope of the present invention is defined by the appended claims.

The object of the invention is to provide a blood-pressure monitoring system that features the following components: 1) a cuffless, wrist-worn blood-pressure monitor featuring a form factor similar to a common watch; 2) a wireless interface that transmits information from the blood-pressure monitor to an Internet-accessible website; and 3) a web services software interface, associated with the Internet-accessible website, that avails information describing blood pressure to other web-based software applications.

The blood-pressure monitor features a watch component with individual sensors that measure optical and pressure waveforms, and a microcontroller that analyzes these waveforms to determine beat-to-beat blood pressure without using a constrictive cuff. A short-range wireless transmitter (using, e.g., a Bluetooth™ protocol) within the watch component sends this information to a matched receiver in the wireless interface. The wireless interface also includes a long-range wireless transmitter (e.g., a radio modem) that sends the blood-pressure information through a wireless network to the Internet-based website.

Web pages rendered by a host computer system display blood pressure and other information using a series of graphs and numerical tables. So that this information can be used by a secondary software application (e.g. a web-based system used in a hospital), the host computer system uses the web services software interface to send data in response to a request.

The web services software interface is based on Extensible Markup Language (XML), a computer language that encloses data in ‘documents’ that are portable between software applications. XML is a system-independent language for representing data. In the present invention, data are sent across a web services software interface in the form of simple object access protocol (SOAP) messages, which are XML-based messages that are transmitted through standard Internet protocols, e.g. hypertext transfer protocol (HTTP). Like hypertext mark-up language (HTML), which is used to code web pages, XML enloses data in ‘tags’ that are interpreted by the receiving software application. However, unlike HTML, the tags disclose the meaning of the enclosed data. XML is extensible, meaning that a user can develop application-specific tags to disclose a wide range of data. In addition, with XML, the user can create a ‘schema’ that describes the structure of the XML document, e.g. which tags are used and where they can occur.

In the present invention, the web services software interface sends information describing cardiac parameters such as time-dependent blood pressure, heart rate, and pulse oximetry in a SOAP message from the host database to a secondary software application. The information is typically sent in response to a query from the secondary software application. In this way, the information is ‘pulled’ rather than ‘pushed’ to the secondary software application. With the web services software interface, the information is formatted to be independent of the secondary software application requesting the data, as well as any ‘downstream’ processing that may occur.

Patients can order the monitor using a separate page in the Internet-based website and it use continuously for a short (e.g. 1 month) period of time. During this time information is periodically sent (e.g., every 15 minutes) to the website, where software monitors the incoming data and transmits summary reports to the patient. When the monitoring period is complete the patient returns the monitor.

Specifically, the invention provides a system for monitoring blood pressure that includes: 1) a gateway software system that receives blood pressure information collected with a blood-pressure monitor and transmitted with a wireless interface; 2) a database that receives the blood pressure information from the gateway software system and stores this information or derivatives thereof; and 3) a web services software interface that, in response to a request from a secondary software system, retrieves the blood pressure information or derivative thereof from the database.

The web services software interface typically includes computer code that processes messages comprising an application-independent format, such as an XML or SOAP format. In addition, the interface typically includes an RPC SOAP servlet that uses computer code to process a SOAP message sent from the secondary software system and extracts at least one parameter from the SOAP message. Once this parameter is extracted, the web services software interface passes it to an EJB that communicates with the database. The EJB typically extracts information from the database, and can be a stateless session bean. The EJB typically includes computer code that can process a WSDL file, send at least one parameter to a SOAP servlet, and send the blood pressure information to the secondary software system.

Both the watch component and the wireless interface typically include a short-range wireless transmitter operating on a wireless protocol based on Bluetooth™, part-15, or 802.11. In this case, ‘part-15’ refers to a conventional low-power, short-range wireless protocol, such as that used in cordless telephones. In typical embodiments, the short-range wireless transmitter sends information to the wireless interface, which is external and typically includes a short-range wireless receiver (also operating a Bluetooth™, part-15, or 802.11 wireless protocol) and a long-range wireless transmitter. The long-range wireless transmitter transmits information over a terrestrial, satellite, or 802.11-based wireless network. Suitable networks include those operating at least one of the following protocols: CDMA, GSM, GPRS, Mobitex, DataTac, iDEN, and analogs and derivatives thereof.

To measure blood pressure, the watch component includes a pressure-monitoring module that generates a pressure waveform, and an optical module that generates an optical waveform. A microprocessor within the watch component runs computer-readable code that processes both the optical and pressure waveforms to determine blood pressure as described in more detail below. The term ‘microprocessor’ means a silicon-based microprocessor or microcontroller that can run compiled computer code to perform mathematical operations on data stored in a memory. Examples include ARM7 or ARM9 microprocessors manufactured by a number of different companies; AVR 8-bit RISC microcontrollers manufactured by Atmel; PIC CPUs manufactured by Microchip Technology Inc.; and high-end microprocessors manufactured by Intel and AMD.

In the above-described system, the term ‘wireless network’ refers to a standard wireless communication network. These networks, described in more detail below, connect a wireless transmitter or a silicon-based chipset to the Internet-based software piece.

The invention has many advantages. In particular, it allows patients to conduct a low-cost, comprehensive, real-time monitoring of their blood pressure. Using the web services software interface, the invention then avails this information to hospitals, home-health care organizations, insurance companies, pharmaceutical agencies conducting clinical trials and other organizations. Information can be viewed using an Internet-based website, a personal computer, or simply by viewing a display on the monitor. Data measured several times each day provide a relatively comprehensive data set compared to that measured during medical appointments separated by several weeks or even months. This allows both the patient and medical professional to observe trends in the data, such as a gradual increase or decrease in blood pressure, which may indicate a medical condition. The invention also minimizes effects of white coat syndrome since the monitor automatically makes measurements with basically no discomfort; measurements are made at the patient's home or work, rather than in a medical office.

With the web services software interface, the system can collect important cardiac information and transfer this to a secondary software application. The transfer process is independent of the nature of the secondary software application, as well as the data format and any processing done by the secondary software application. This means that the system acts essentially as a ‘data provider’ to many other software applications, each of which can carry out a different function. Blood pressure is known to be perhaps the most important metric describing a patient's cardiac health, and, with the invention, this information can be measured in real time and easily transferred to a wide range of secondary software applications for further analysis.

Real-time, automatic blood pressure measurements, followed by wireless transmission of the data, are only practical with a non-invasive, cuffless monitor like that of the present invention. Measurements can be made completely unobtrusive to the patient. And the monitor alleviates conditions, such as an uncomfortable or poorly fitting cuff, that can erroneously affect a blood-pressure measurement.

The monitor can also measure pulse oximetry to characterize the patient's heart rate and blood oxygen saturation using the same optical system for the blood-pressure measurement. These data can be wirelessly transmitted and used to further diagnose the patient's cardiac condition.

The monitor is small, easily worn by the patient during periods of exercise or day-to-day activities, and non-invasively measures blood pressure in a matter of seconds without affecting the patient. An on-board or remote processor can analyze the time-dependent measurements to generate statistics on a patient's blood pressure (e.g., average pressures, standard deviation, beat-to-beat pressure variations) that are not available with conventional devices that only measure systolic and diastolic blood pressure at isolated times.

Ultimately, the wireless, internet-based blood pressure-monitoring system described herein provides an in-depth, cost-effective mechanism to evaluate a patient's cardiac condition. Certain cardiac conditions can be controlled, and in some cases predicted, before they actually occur. Moreover, data from the patient can be collected and analyzed while the patient participates in their normal, day-to-day activities. This provides a relatively comprehensive diagnosis that is not possible using a conventional medical-diagnostic system.

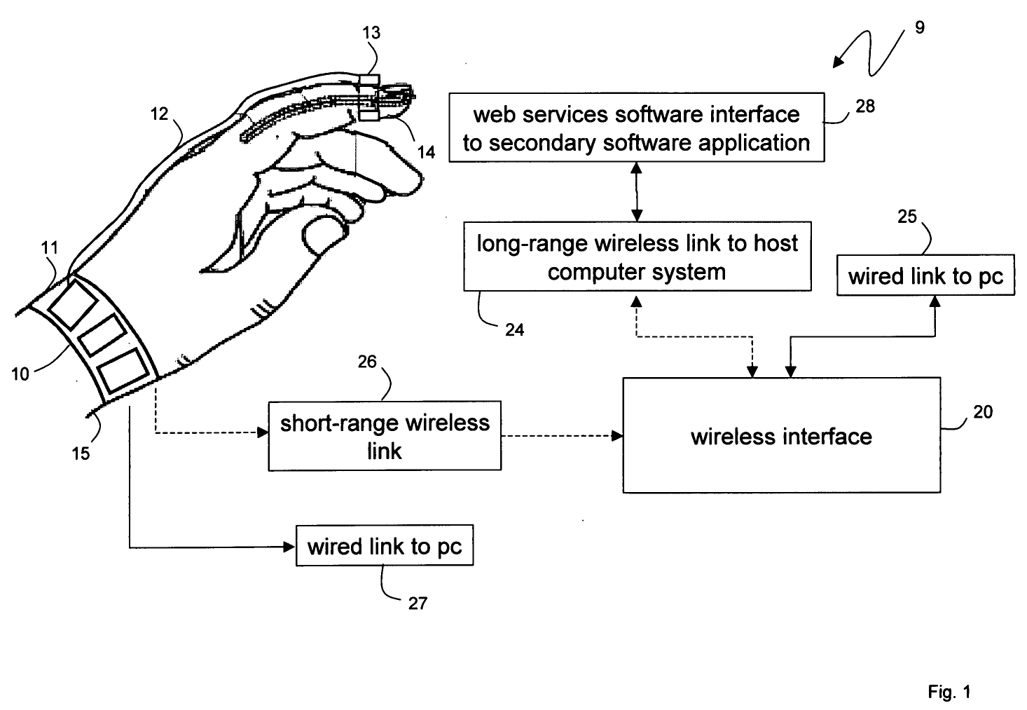

FIG. 1 shows a cuffless blood-pressure monitoring system 9 according to the invention that measures a patient's real-time, beat-to-beat blood pressure. The system 9 features a watch component 10 that measures blood pressure without using a cuff, and a wireless interface 20 that receives and transmits this information through a long-range wireless link 24 to a host computer system. The host computer system, in turn, includes a web services software interface 28 that sends information through the Internet to a secondary software system. Using the web services software interface 28, information can also be sent from the secondary software system, through the long-range wireless link 24 and wireless interface 20, to the watch component 10. This information, for example, can be used to change a property of the watch component (forcing it, e.g., to collect blood pressure readings at a higher rate), or send a text message.

The watch component 10 features an optical finger-mounted module 13 that attaches to a patient's index finger 14, and a wrist-mounted module 11 that attaches to an area 15 of the patient's wrist where a watch is typically worn. A cable 12 provides an electrical connection between the finger-mounted 13 and wrist-mounted 11 modules. During operation, the finger-mounted module 13 measures an optical ‘waveform’ and the wrist-mounted module measures a pressure ‘waveform’ as described in detail in the following pending patent applications, filed with this application, the contents of which are incorporated by reference: CUFFLESS SYSTEM FOR MEASURING BLOOD PRESSURE (U.S. Ser. No.______) and CUFFLESS BLOOD-PRESSURE MONITOR AND ACCOMPANYING WIRELESS, INTERNET-BASED SYSTEM (U.S. Ser. No.______).

Once these waveforms are measured, the watch component 10 processes them to determine diastolic and systolic blood pressure, real-time beat-to-beat blood pressure, heart rate, and pulse oximetry. The watch component 10 transfers this information using a short-range wireless link 26 to the wireless interface 20. The interface 20 receives the information and, in turn, sends it over the long-range wireless link 24 to an Internet-accessible website. In order to send information directly to a personal computer, both the watch component 10 and the wireless interface 20 include wired links 25, 27 (e.g., a serial cable connected to a serial port) to a personal computer.

Software programs associated with the Internet-accessible website, secondary software system, and the personal computer analyze the blood pressure, and heart rate, and pulse oximetry values to characterize the patient's cardiac condition. These programs, for example, may provide a report that features statistical analysis of these data to determine averages, data displayed in a graphical format, trends, and comparisons to doctor-recommended values.

The blood-pressure monitor 9 measures cardiac information non-invasively with basically no inconvenience to the patient. This means information can be measured in real time and throughout the day, e.g., while the patient is working, sleeping, or exercising. For example, during work or sleep, the wireless interface 20 rests near the patient (e.g. on a desktop), while during exercise it attaches to the patient's belt. In this way, the blood-pressure monitor 9, combined with the above-described software programs, provides an extensive, thorough analysis of the patient's cardiac condition. Such analysis is advantageous compared to conventional blood-pressure measurements, which are typically made sporadically with an uncomfortable cuff, and thus may not accurately represent the patient's cardiac condition.

When a distance greater than twenty feet separates the interface 20, the watch component 10 simply stores information in memory and continues to make measurements. The watch component automatically transmits all the stored information (along with a time /date stamp) when it comes in proximity to the interface 20, which then transmits the information through the wireless network.

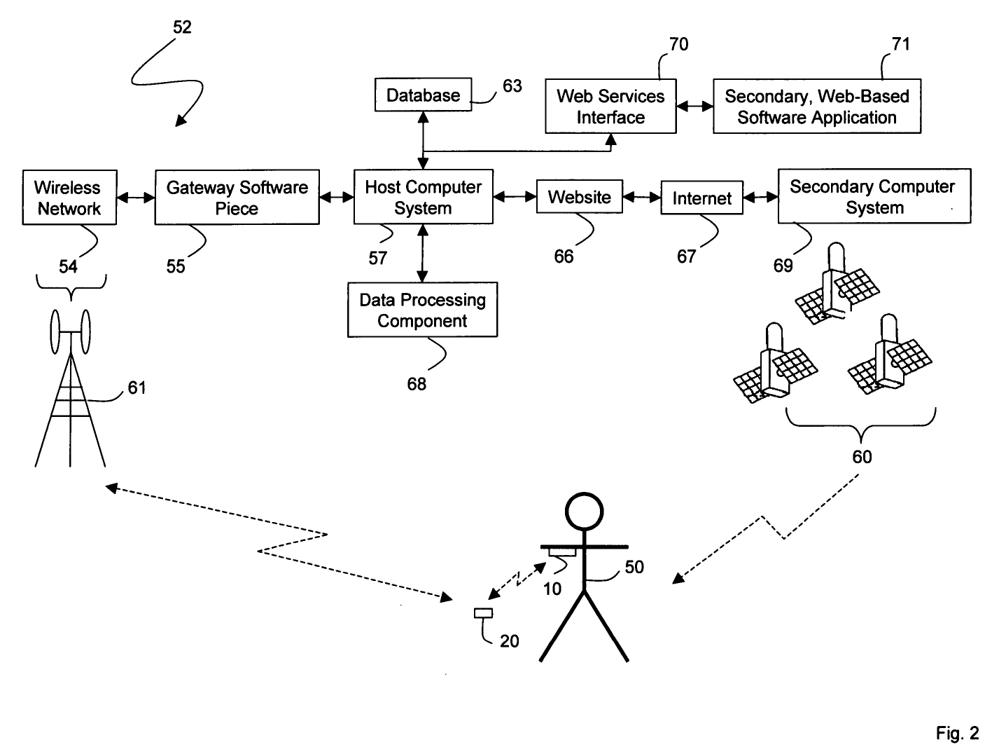

FIG. 2 shows an Internet-based system 52 that operates in concert with the watch component 10 and wireless interface 20 to send information from a patient 50 through a two-way wireless network 54 to an Internet-based host computer system 57. The host computer system 57 hosts a web site 66 that can be accessed by a secondary computer system 69 through the Internet 67. The system 52 functions in a bi-directional manner, i.e. the wireless interface 20 can both send and receive data. Most data flows from the interface 20; using the same network, however, this module also receives data (e.g., ‘requests’ to measure data or text messages) and software upgrades.

The host computer system 57 additionally includes a database 63 and a data-processing component 68 for, respectively, storing and analyzing the data. The host computer system 57, for example, may include multiple computers, software pieces, and other signal-processing and switching equipment, such as routers and digital signal processors. The host computer system 57 also hosts the web site 66 using conventional computer hardware (e.g. computer servers for both a database and the web site) and software (e.g., web server and database software).

Data are typically transmitted through the wireless network 54 as packets that feature both a ‘header’ and a ‘payload’. The header includes an address of the source wireless transmitter and a destination address on the network. The payload includes the above-described data. Data packets are transmitted over a conventional wireless terrestrial network, such as a CDMA, GSM/GPSRS, Mobitex, or DataTac network. Or they may be transmitted over a satellite network, such as the Orbcomm network. The specific network is associated with the wireless transmitter used by the wireless interface to transmit the data packet.

A gateway software piece 55 connects to the wireless network 54 and receives data packets from one or more devices by connecting to the wireless network 54 using a TCP/IP-based connection, or with a dedicated, digital leased line (e.g., a frame-relay circuit or a digital line running an X.25 protocol).

The web services software interface 70 connected to the host computer system sends information using an XML-based web services link to a secondary, web-based software application 71. For example, this application could be used by a home-health care organization to remotely monitor patients at their homes. Or it could be used by a hospital to receive and display blood pressure and other information measured from their patients outside of the hospital. In yet another application, the secondary software application could be used by a pharmaceutical company managing a clinical trial to collect, display, and analyze blood pressure information from a number of different patients.

As described above, using the web services interface 70, the secondary software system 71 typically ‘pulls’ the data from the database 13, as opposed to having the data ‘pushed’ to it. The secondary software system 15 is typically designed on a software platform that supports web services, e.g. the Java 2 Platform, Enterprise Edition (J2EE™) or Microsoft's ‘.Net’ platform. Secondary software systems built on J2EE™ can connect to other software applications through web services, and include essential features such as security, distributed transaction management, and connection pool management.

The web services software interface communicates with secondary software systems that can be either end-user applications (e.g., a web site), or software systems that are also based on web services. The secondary software system can use the blood pressure and pulse oximetry information by itself, or combine and processes this information with other information (e.g., a patient's medical records) from other software systems. The other information can be stored directly on the secondary software system, or too can be accessed using a web services software interface.

Typical web services can be implemented with software systems such as BEA WebLogic Server, described in more detail in www.bea.com, the contents of which are incorporated herein by reference. These software systems typically contain a software application, called a servlet, which sends and receives XML-based SOAP messages to and from the secondary software system. The servlet implements ‘remote procedure calls’, or RPCs, between the web services software interface and the secondary software system. Using an RPC, the secondary software system can initiate an action (using, e.g., a mouse click or an automated HTTP request), which then polls data from the database 13 using the web services interface.

To generate blood pressure information for the web services software interface, the patient continuously wears the blood-pressure monitor for a short period of time, e.g. one to two weeks after visiting a medical professional during a typical ‘check up’ or after signing up for a short-term monitoring program through the website. In this case, the watch component 10 measures blood pressure in a near-continuous manner, e.g. every fifteen minutes. This information is then immediately transmitted by the wireless interface 20. For longer-term monitoring, the patient may measure blood pressure once each day for several months.

To view information sent from the blood-pressure monitor, the patient or medical professional accesses a patient user interface hosted on the web site 66 through the Internet 67 from a secondary computer system 69. The patient interface displays blood pressure and related data measured from a single patient. The system 52 may also include a call center, typically staffed with medical professionals such as doctors, nurses, or nurse practioners, whom access a care-provider interface hosted on the same website 66. The care-provider interface displays blood pressure data from multiple patients.

The wrist-worn component 10 may additionally include a GPS that receives GPS signals from a constellation of GPS satellites 60 and processes these signals to determine a location (e.g., latitude, longitude, and altitude) of the monitor and, presumably, the patient. This location could be plotted on a map within the web site 66, and used to locate a patient during an emergency, e.g. to dispatch an ambulance.

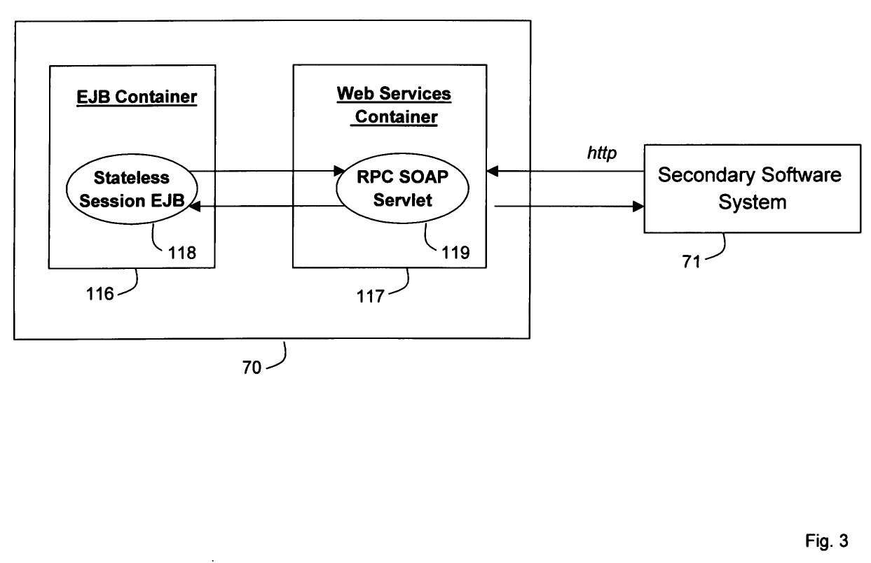

FIG. 3 schematically shows the secondary software system 71 and web services software interface 70 in more detail. The software interface 70 features a web services ‘container’ 117, which is a software application (written, e.g., in Java) developed on a platform such as BEA WebLogic. The container 117 runs an RPC SOAP servlet 119 that communicates over HTTP with the secondary software system 71. A stateless session enterprise java bean (EJB) 118 runs in a software application called the EJB container 116 and implements the RPC SOAP servlet 119. In this way, the servlet 119 appears as a remote object that performs a well-defined function to the secondary software application 71.

When the secondary software system 71 invokes the web service, it sends blood pressure information in the form of a SOAP message to the web services container 117, which then executes the RPC SOAP servlet 119 in response. The servlet 119 then returns parameters to the secondary software system 71 in the form of a second SOAP message.

Typically the web services container 117 includes multiple SOAP servlets, each of which handle different requests in the form of SOAP messages. During operation, the SOAP servlet 119 receives the request formatted in the SOAP message and ‘unwraps’ the message to identify parameters sent from the secondary software system. The SOAP servlet 119 processes the parameters to identify the appropriate stateless session EJB 118 to implement. Once this is done, the SOAP servlet 119 attaches the parameters to the appropriate Java objects within the EJB container 116 and passes the parameters to the corresponding stateless session EJB 118. In response, the stateless session EJB 118 processes the parameter and returns a value. The SOAP servlet 119 generates another SOAP message that includes the return value, and sends this message back to the secondary software system over HTTP. The secondary software system 71 can then display the return value using, e.g., a web site, or integrate the return data into its own database.

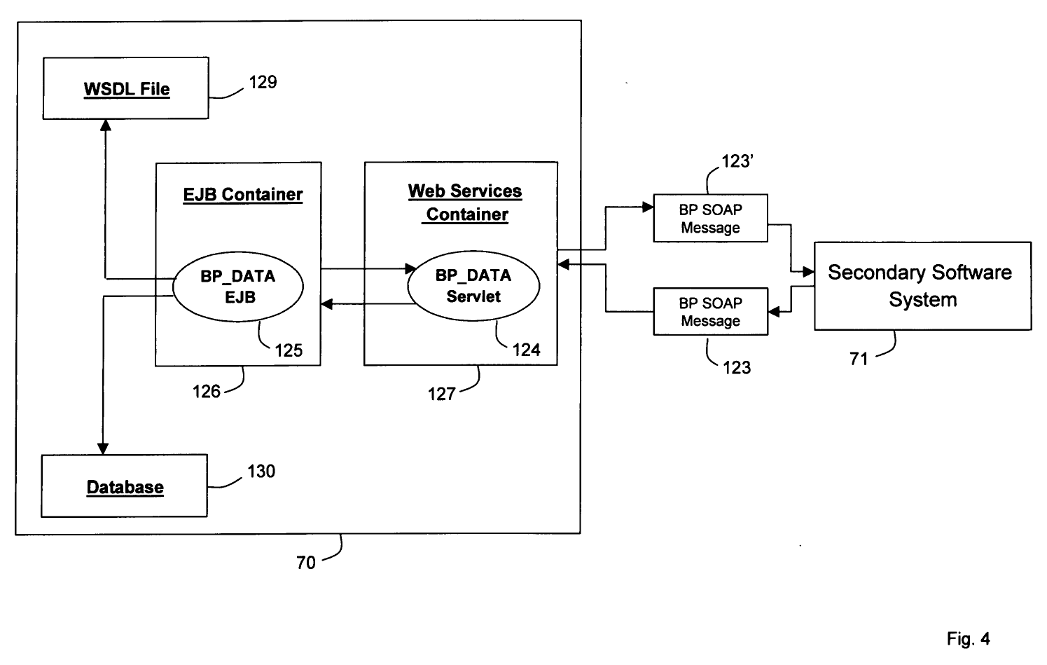

FIG. 4 shows a schematic example of a specific web services operation conducted by a secondary software system 171. In the web service, the secondary software system 171 requests blood pressure information through a web services software interface 170 that includes a web services container 127 and an EJB container 126, similar to those described above with reference to FIG. 3. To process the request, the web services container 127 receives a BP SOAP message 123 from the secondary software system 171 requesting blood pressure information. In response, the web services container 127 initiates a BP_DATA SOAP servlet 124, which unwraps the BP SOAP message 123 and extracts parameters that identify the BP_DATA EJB 124 that needs to be implemented.

The specific BP_DATA web service is defined by a web services description language (WSDL) file 129, which is served dynamically through the BP_DATA EJB 25. The WSDL file 129 is a document, written in XML, which describes the web service for retrieving blood pressure readings. It specifies the location (i.e. computer-based address) of the service, and the operations that it conducts.

The BP_DATA SOAP servlet 124 invokes the BP_DATA EJB 125 and includes all the necessary information to carry out a request. This information includes, for example: a serial number of the watch monitor that measures the data; the hospital associated with the patient; and the name, username, and password of the patient associated with the watch component. The BP_DATA EJB 125 sends a query to a database 130 to authorize the request by ensuring that the requestor (i.e. the user) has a valid username and password. Once the user is authenticated, the EJB 125 then queries the database 130 again and generates a response ‘string’ that is an XML document. The document includes either the requested blood pressure information, or an ‘error statement’ indicating that the information is not present. The EJB 125 then returns the XML document as a‘payload’ to the SOAP servlet 124, which returns it as another SOAP message 123′ to the secondary software system 71.

The web service is said to be ‘complete’ when it processes the request by the secondary software system 71, and this system receives the associated SOAP message 123′. At this point, the secondary software system 71 parses an XML payload within the SOAP message and incorporates the blood pressure information into an application, e.g. a web-based system for a hospital. If the XML payload includes an error statement then the web site renders an error message.

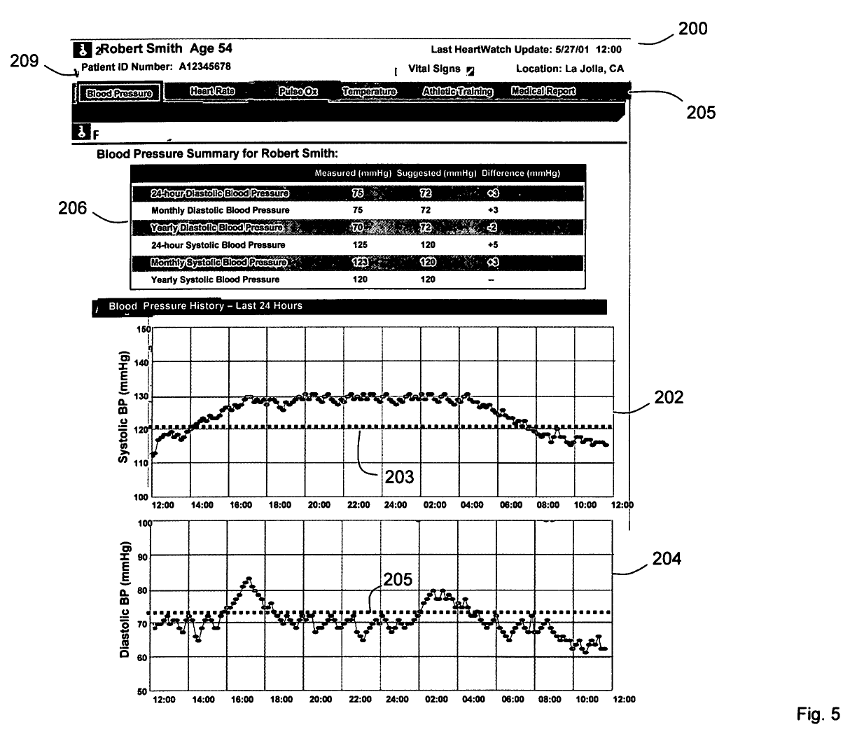

FIG. 5 shows a web page 200 that could be used by the secondary software application described above. The web page 200 includes a header field 209 that lists general information about the patient (e.g. name, age, and ID number, general location, and information concerning recent measurements); a table 206 that lists recently measured blood pressure data and suggested (i.e. doctor-recommended) values of these data; and graphs that plot the systolic 202 and diastolic 204 blood pressure data in a time-dependent manner. The header field additionally includes a series of tabs 205 that each link to separate web pages that include, e.g., tables and graphs corresponding to a different data measured by the watch component. These include heart rate, pulse oximetry, and temperature. The tabs 205 also link to web pages that display the patient's GPS-determined location and a detailed medical report.

The table 206 lists a series of data fields that show running average values of the patient's daily, monthly, and yearly systolic and diastolic blood pressure levels. The levels are compared to a series of corresponding ‘suggested’ values of systolic pressure that are extracted from a database associated with the web site. The suggested values depend on, among other things, the patient's age, sex, and weight. The table then calculates the difference between the running average and suggested values to give the patient an idea of how their data compares to that of a healthy patient.

The graphs 202, 204 show plots of the patient's systolic and diastolic blood pressure vs. a time stamp corresponding to a particular measurement. These time-dependent data are then compared to lines 203, 205 indicating the same suggested values (in this case 120 and 72 mmHg) of systolic blood pressure listed in the table 206. The graphs 202, 204 show trends in the blood pressure levels that, for example, may be used to adjust the patient's diet, exercise level, or medication.

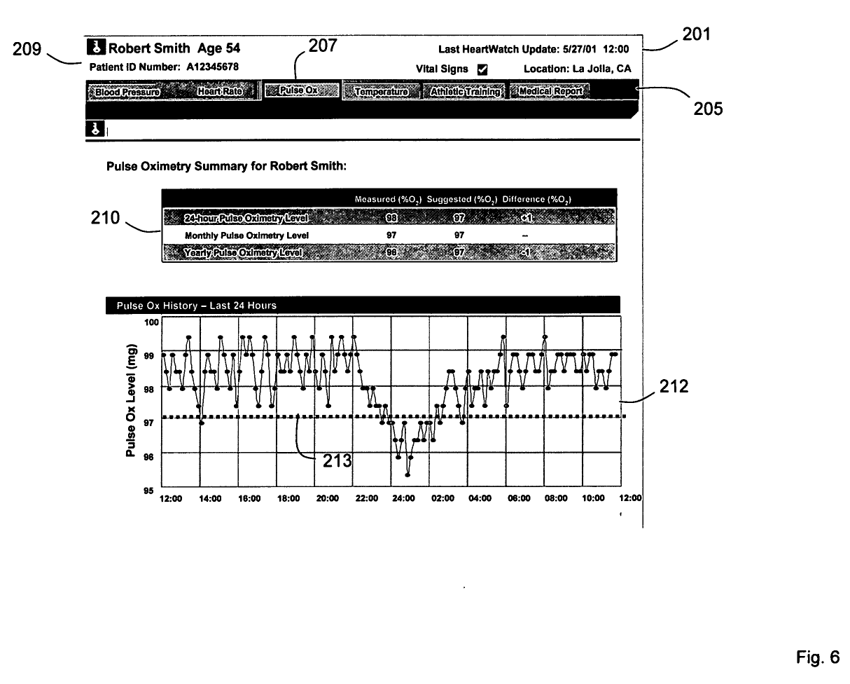

FIG. 6 shows a web page 201 that is accessed by clicking on a tab 207 entitled ‘Pulse Ox’ that displays information describing the patient's pulse oximetry. The web page 201 includes a table 210 comparing daily, monthly, and yearly averages of pulse oximetry to their suggested value; a graph 212 showing time-dependent pulse oximetry values compared to a line 213 indicating the suggested value; and the same header field 209 shown in FIG. 4.

Other embodiments are within the scope of the invention. For example, the web services software interface can feature a suite of web services that are ‘message’ based or asynchronous in nature. This is commonly called a ‘loosely coupled’ web service and would replace the above-described system using an RPC SOAP servlet. Loosely coupled web services permit for a ‘conversation’ to take place between the secondary software system and the software system with the web services software interface. Such a system could be used for, e.g., sending a first message to a watch component to take a measurement, followed by sending a second message to the watch component to request a status of this operation.

The web services software interface may also include security measures such as authentication, authorization, encryption, credential presentation, and digital signature resolution. The interface may also be modified to conform to industry-mandated, XML schema definitions, while being ‘backwards compatible’ with any existing XML schema definitions.

In still other embodiments, the web services software interface is designed to be interoperable with other web services implementations, such as Microsoft Net and IBM Websphere.

The blood-pressure monitoring system can also be used in ways other than those described above. For example, in one embodiment, a patient using an Internet-accessible computer and web browser, such as those described in FIG. 2, directs the browser to an appropriate URL and signs up for a service for a short-term (e.g., 1 month) period of time. The company providing the service completes an accompanying financial transaction (e.g. processes a credit card), registers the patient, and ships the patient a blood-pressure monitor for the short period of time. The registration process involves recording the patient's name and contact information, a number associated with the monitor (e.g. a serial number), and setting up a personalized website. The patient then uses the monitor throughout the monitoring period, e.g. while working, sleeping, and exercising. During this time the monitor measures data from the patient and wirelessly transmits it through the channel described in FIG. 2 to a data center. There, the data are analyzed using software (e.g., reporting software supported by an Oracle™ database) running on computer servers to generate a statistical report. The computer servers then automatically send the report to the patient using email, regular mail, or a facsimile machine at different times during the monitoring period. When the monitoring period is expired, the patient ships the blood-pressure monitor back to the monitoring company.

Web pages used to display the data can take many different forms, as can the manner in which the data are displayed. Web pages are typically written in a computer language such as HTML, and may also contain computer code written in languages such as java and javascript for performing certain functions (e.g., sorting of names). The web pages are also associated with database software (provided by companies such as Oracle and Microsoft) that is used to store and access data. Equivalent versions of these computer languages and software can also be used. In general, the graphical content and functionality of the web pages may vary substantially from what is shown in the above-described figures. In addition, web pages may also be formatted using standard wireless access protocols (WAP) so that they can be accessed using wireless devices such as cellular telephones, personal digital assistants (PDAs), and related devices.

Different web pages may be designed and accessed depending on the end-user. As described above, individual users have access to web pages that only their blood pressure data (i.e., the patient interface), while organizations that support a large number of patients (e.g. hospitals) have access to web pages that contain data from a group of patients (i.e., the care-provider interface). Other interfaces can also be used with the web site, such as interfaces used for: insurance companies, members of a particular company, clinical trials for pharmaceutical companies, and e-commerce purposes. Blood pressure data displayed on these web pages, for example, can be sorted and analyzed depending on the patient's medical history, age, sex, medical condition, and geographic location.

The web pages also support a wide range of algorithms that can be used to analyze data once they are extracted from the data packets. For example, an instant message or email can be sent out as an ‘alert’ in response to blood pressure indicating a medical condition that requires immediate attention. Alternatively, the message could be sent out when a data parameter (e.g. systolic blood pressure) exceeds a predetermined value. In some cases, multiple parameters (e.g., blood pressure and pulse oximetry) can be analyzed simultaneously to generate an alert message. In general, an alert message can be sent out after analyzing one or more data parameters using any type of algorithm. These algorithms range from the relatively simple (e.g., comparing blood pressure to a recommended value) to the complex (e.g., predictive medical diagnoses using ‘data mining’ techniques). In some cases data may be ‘fit’ using algorithms such as a linear or non-linear least-squares fitting algorithm. In general, any algorithm that processes data collected with the above-described method is within the scope of the invention.

In other embodiments, the blood-pressure monitoring device, such as that shown in FIG. 1, sends information directly to a personal computer, where it is then processed and analyzed. For example, the personal computer can run a client-side application that processes the information and displays it in a manner that is similar to that described with reference to FIGS. 5 and 6. The client-side application can then send information through the Internet using HTTP to a secondary software application.

Still other embodiments are within the scope of the following claims.

1. A system for monitoring blood pressure comprising:
a gateway software system that receives blood pressure information collected with a blood-pressure monitor and transmitted with a wireless interface;
a database that receives the blood pressure information from the gateway software system and stores this information or derivatives thereof; and
a web services software interface that, in response to a request from a secondary software system, retrieves the blood pressure information or derivative thereof from the database.

2. The system of claim 1, wherein the web services software interface further comprises computer code that processes messages comprising an application-independent format.

3. The system of claim 2, wherein the application-independent format is an XML format.

4. The system of claim 2, wherein the application-independent format is a SOAP format.

5. The telematics system of claim 1, wherein the web services software interface comprises an RPC SOAP servlet.

6. The telematics system of claim 5, wherein the RPC SOAP servlet comprises computer code that processes a SOAP message sent from the secondary software system.

7. The telematics system of claim 6, wherein the RPC SOAP servlet further comprises computer code that extracts at least one parameter from the SOAP message.

8. The telematics system of claim 7, wherein the web services software interface is configured to pass the parameter to an enterprise Java bean.

9. The telematics system of claim 1, wherein the web services software interface further comprises at least one enterprise Java bean.

10. The telematics system of claim 9, wherein the enterprise Java bean comprises computer code that communicates with the database.

11. The telematics system of claim 10, wherein the enterprise Java bean further comprises computer code that extracts information from the database.

12. The telematics system of claim 11, wherein the enterprise Java bean is a stateless session bean.

13. The telematics system of claim 8, wherein the enterprise Java bean comprises computer code that processes a WSDL file.

14. The telematics system of claim 8, wherein the enterprise Java bean further comprises computer code that sends at least one parameter to a SOAP servlet.

15. The telematics system of claim 1, wherein the web services software interface further comprises computer code to send the blood pressure information to the secondary software system.

16. The telematics system of claim 15, wherein the web services software interface further comprises computer code to send an XML message comprising blood pressure information to the secondary software system.

17. The telematics system of claim 15, wherein the web services software interface further comprises computer code to send a SOAP message comprising blood pressure information to the secondary software system.

18. A telematics system comprising:
a gateway software system that receives blood pressure information transmitted wirelessly from a body-worn device;
a database that receives the blood pressure information from the gateway software system and stores this information or derivatives thereof; and
a web services software interface that, in response to a request from a secondary software system, retrieves the blood pressure information or derivative thereof from the database.
